(19) United States
(12) Patent Application Publication
(10) Pub. No.: US 2004/0235715 A1
(43) Pub. Date: Nov. 25, 2004

(54) Method of producing glycosylated bikunin

(75) Inventors: Sham-Yuen Chan, El Sobrante, CA (US); Teresa Mo-fun Wong, Lafayette, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Bayer Corporation

(21) Appl. No.: 10/874,850

(22) Filed: Jun. 22, 2004

Related U.S. Application Data
(62) Division of application No. 09/441,654, filed on Nov. 12, 1999.

(51) Int. Cl. A61K 38/17; C07H 21/04; C07K 14/47

(52) U.S. Cl. 514/8; 435/69.1; 435/320.1; 435/325; 530/395; 536/23.5

(57) ABSTRACT

A method for the production of bikunin or monokunin from mammalian cell culture is described. The product exhibits glycosylation characteristic of proteins produced from mammalian cells. The method is used in a preferred example to produce a truncated human placental bikunin.

### RELATED APPLICATION

This application contains material related to U.S. patent application Ser. No. 09/144,428 to Tamburini et al., filed Aug. 31, 1998, entitled “Human Bikunin,” which is incorporated herein by reference.

### BACKGROUND OF THE INVENTION

1. Field

This invention generally describes a production method for recombinant therapeutic proteins having a human-like glycosylation pattern. Specifically, the invention is directed to glycosylated bikunin capped with sialic acids having alpha-(2, 3) and alpha-(2,6) linkages.

2. Background

Protein inhibitors are classified into a series of families based on extensive sequence homologies among the family members and the conservation of intrachain disulfide bridges (for review, see Laskowski and Kato, Ann. Rev. Biochem. 49: 593-626, 1980). Serine protease inhibitors of the Kunitz family are characterized by their homology with aprotinin (bovine pancreatic trypsin inhibitor). Aprotinin is known to inhibit various serine proteases including trypsin, chymotrypsin, plasmin and kallikrein. Kunitz-type inhibitor domains have been reported in larger proteins such as the inter-α-trypsin inhibitors (Hochstrasser et al., Hoppe-Seylers Z. Physiol. Chem. 357: 1659-1661, 1969 and Tschesche et al., Eur. J. Biochem. 16: 187-198, 1970), the β-amyloid protein precursor and the α3-collagen type VI (Chu et al., EMBO J. 9: 385-393, 1990). TFPI (also known as extrinsic pathway inhibitor (EPI) or lipoprotein-associated coagulation inhibitor (LACI)) is a plasma protease inhibitor that consists of three tandem Kunitz-type inhibitors flanked by a negatively charged amino terminus and a positively charged carboxyl terminus. The first and second Kunitz-type domains have been shown to inhibit factor VIIa and factor Xa activity, respectively. Bikunin is a two Kunitz domain human homologue of bovine aprotinin (Delaria et al, J. Biol. Chem. 272(18): 12209-12214, 1997; Marlor et al, J. Biol. Chem. 272(18): 12202-12208, 1997).

Placental bikunin, a novel human serine protease inhibitor containing two Kunitz-like domains, has been cloned and expressed (Delaria et al, J. Biol. Chem. 272(18): 12209-12214, 1997). Characterization studies showed that truncated placental bikunin is a potent inhibitor of kallikrein and plasmin. The sequence of truncated placental bikunin is shown in FIG. 2. The protease inhibitory function of bikunin suggests that placental bikunin has important therapeutic application for the treatment of a variety of disorders including prevention of disseminated intravascular coagulation, reduction of blood loss during surgery, reduction of brain edema following vascular injury, and blockage of tumor growth and invasiveness (Marlor et al, J. Biol. Chem. 272(18): 12202-12208, 1997). An unexpected observation was made recently that placental bikunin was able to increase airway surface liquid osmolarity and mucociliary transport in animal models (U.S. patent application Ser. No. 09/144,428 to Tamburini et al., filed Aug. 31, 1998, entitled “Human Bikunin.”) Thus there is a need to produce placental bikunin in large quantities.

U.S. Pat. No. 5,914,315 to Sprecher et al. (Jun. 22, 1999) describes a protein possessing a Kunitz-type domain with putative protease inhibitor activity. Sprecher also describes DNA molecules coding for said protein and host cells expressing the protein.

Despite numerous attempts, high level expression of bikunin in E. coli and yeast has been unsuccessful, and such expression fails to yield protein having mammalian- or human-like glycosylation patterns. Placental bikunin has been produced from insect cells in culture. Proteins expressed from insect cells have been known to lack complex oligosaccharides terminating with sialic acid and to have a significant reduction in plasma half-life (Grossman et al., Endocrinology 138: 92-100, 1997). Although insect-derived proteins are bioactive in vitro, they are not suitable for clinical use due to the altered glycosylation. It is also known that sialyltransferase activity is not present in baculovirus-infected insect cells (Hooker et al Biotechnology and Bioengineering 63: 559-572, 1999).

It is well recognized that the biological activity of a glycoprotein is dependent upon not only the integral structure of the protein, but also the properties of the oligosaccharide covalently attached to the protein. Glycosylation can effect solubility, resistance to proteolytic attack and thermal inactivation, quaternary structure, activity, targeting, antigenicity, functional activity, and half-life of the protein. Mammalian glycosylation patterns are well known and are described in Fukuda et al. (1994), Molecular Glycobiology, IRL Press, New York, incorporated herein by reference. Sialic acid has been shown to be important in sustaining the half-life of protein therapeutics. It is known that desialylated or under-sialylated glycoproteins have significantly reduced half-life in plasma. Thus it is advantageous to produce sialylated placental bikunin using mammalian cells.

### SUMMARY OF THE INVENTION

This invention describes a method for the production of glycosylated placental bikunin. Preferably the cell host is CHO cells, but the production can be done with other cell hosts including HKB cells (see U.S. patent application Ser. No. 09/209,920 to Cho filed Dec. 10, 1998, incorporated herein by reference), myeloma, and 293S cells. The production medium is preferably a chemically-defined medium free of plasma protein supplements.

This invention is illustrated with a truncated placental bikunin having the amino acid sequence shown in FIG. 2. As used herein, “bikunin” refers to any protein which has two Kunitz domains. See Laskowski et al., 1980, Ann Rev. Biochem. 49: 593-626. Kunitz-type inhibitors have been described in the patent literature. See Sprecher et al., U.S. Pat. No. 5,914,315 (Jun. 22, 1999). Kunitz domains that exist within larger proteins have been shown to retain their functional activities when produced as single domains (Delaria et al., 1997, J. Biol. Chem. 272:12209-12). Thus, the method of producing glycosylated bikunin disclosed herein is expected to extend to the production of glycosylated polypeptides having only one Kunitz domain (denoted “monokunins”). Such polypeptides having only one Kunitz domain and having mammalian-like glycosylation are considered to be within the scope of the present invention. The monokunin may be derived from or may be a fragment of a known bikunin. Modification of DNA constructs (described herein) used to express the glycosylated polypeptides may be accomplished by substituting the desired monokunin coding sequence for the bikunin coding sequence. Such modification is within the capability of one of skill in the art.

The present invention encompasses a method for the production of glycosylated bikunin, preferably glycosylated bikunin having at least 80% identity over 40 residues to SEQ ID NO: 1 more preferably glycosylated bikunin having at least 85% identity over 45 residues to SEQ ID NO: 1, still more preferably glycosylated bikunin having at least 90% identity over 50 residues to SEQ ID NO: 1, even more preferably glycosylated bikunin having at least 95% N identity over 55 residues to SEQ ID NO: 1. The method also extends to the production of glycosylated monokunins, preferably glycosylated monokunins having at least 80% identity over 40 residues to SEQ ID NO: 1, more preferably glycosylated monokunin having at least 85% identity over 45 residues to SEQ ID NO: 1, still more preferably glycosylated monokunin having at least 90% identity over 50 residues to SEQ ID NO: 1, even more preferably glycosylated monokunin having at least 95% identity over 55 residues to SEQ ID NO: 1.

The glycosylated bikunin produced from the mammalian cells is preferably capped with sialic acids having alpha-(2, 3) and alpha-(2,6) linkages. In a less preferred embodiment, the glycosylation includes sialic acids with only alpha-(2-3) linkages.

### BRIEF DESCRIPTION OF THE FIGURES

FIG. 1 shows the plasmid map of pBC-BK (CMV-IE=cytomegalovirus immediate early; DHFR=dihydrofolate reductase; Amp-r=ampicillin resistance).

FIG. 2 lists the amino acid sequence of truncated placental bikunin.

### DETAILED DESCRIPTION

In a preferred embodiment of present invention, a polypeptide which exhibits a mammalian-like glycosylation has a deduced amino acid sequence which is strongly homologous to SEQ ID NO: 1. In another preferred embodiment the polypeptide of the present invention may include additional amino acid sequences appended to the N- and/or C-terminal of the deduced amino acid sequence given by SEQ ID NO: 1. The polypeptides of the present invention may be derived from a recombinant source or a natural source, and are preferably of recombinant origin. As used herein, “protein” is synonymous with “polypeptide.”

The present invention further includes a polypeptide which shares at least a 60%, more preferably at least an 80%, still more preferably a 90%, or most preferably at least a 95% sequence identity over at least 20, more preferably at least 30, still more preferably at least 40, or most preferably at least 50 residues with SEQ ID NO: 1. (Such polypeptides may be herein referred to as “polypeptides of the present invention”.) In one embodiment the polypeptide of the present invention is characterized by having a deduced amino acid sequence given by SEQ ID NO: 1. In another embodiment of the invention, the polypeptide of the present invention comprises the amino acid sequence of SEQ ID NO: 1 and further comprises at least one additional amino acid sequence appended to the N- and/or C-terminal of SEQ ID NO: 1.

In more preferred embodiments, the polypeptides described above have a mammalian-like glycosylation pattern. The oligosaccharide chains of glycoproteins are classified into 2 groups (Kobata 1995 in Molecular Biology and Biotechnology, edited by R. Meyers, p 383-385, Wiley-VCH, incorporated herein by reference): O-linked oligosaccharide chains and N-linked oligosaccharide chains. All O-linked sugar chains of the mucin type contain N-acetylgalactosamine residues at their reducing termini, which are linked to the hydroxyl groups of Ser and Thr residues of polypeptide chains. In contrast, all N-linked sugar chains contain N-acetylglucosamine residues at their reducing termini, which are linked to the amide groups of Asn residues in the Asn-X-Thr/Ser sequence in the polypeptide chains. Both groups of sugar chains contain complex structural variations. However, mammalian cells produce proteins having sugar chains capped with sialic (neuraminic) acid residues. As used herein, a mammalian glycosylated bikunin is any bikunin having a mammalian-like glycosylation profile, or a glycosylation profile similar to that of native proteins produced by mammalian cells. As used herein, a mammalian glycosylated monokunin is any monokunin having a mammalian-like glycosylation profile, or a glycosylation profile similar to that of native proteins produced by mammalian cells.

Such a polypeptide as described above may be (i) one in which one or more of the amino acid residues are substituted (relative to SEQ ID NO: 1) with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethyleneglycol), or (iv) one in which additional amino acids are fused to the mature polypeptide, such as a leader or secretory sequence or a sequence which is employed for purification of the mature polypeptide or a proprotein sequence or mature protein sequence beyond the sequence given in SEQ ID NO: 1, or (v) one in which one or more amino acids are deleted from or inserted into the sequence of the polypeptide. Combinations of the above-described types of variations in the peptide sequence are within the scope of the invention. Such polypeptides are deemed to be within the scope of those skilled in the art from the teachings herein.

The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

The current invention encompasses mammalian cells in culture which express a polypeptide of the present invention. Polypeptides of the present invention are preferably provided in an isolated form, and preferably are purified to homogeneity. The term “isolated” means that the material is removed from its original environment (e.g., the natural environment if it is naturally occurring). For example, a naturally occurring polypeptide present in a living animal is not isolated, but the same polypeptide, separated from some or all of the coexisting materials in the natural system, is isolated. Such polypeptide could be part of a composition and still be isolated in that such composition is not part of its natural environment.

As used herein “identity” between two polypeptides is determined by comparing the amino acid sequence of one polypeptide to the sequence of a second polypeptide. “Identity” refers to sequence conservation, or “homology”, between two or more peptides or two or more nucleic acid molecules, normally expressed in terms of percentage identity over a given number of residues. When a position in the compared sequences is occupied by the same base or amino acid (“residue”), then the molecules are identical at that position. The percent identity can be maximized by aligning the compared sequences alongside each other, sliding them back and forth, and conservatively introducing gaps in the sequences where necessary. The percent identity is calculated by counting the number of identical aligning residues, dividing by the total length of the aligned region, including gaps in both sequences, and multiplying by 100. Identity would thus be expressed as, e.g., “60% identity over 200 amino acids,” or “57% identity over 250 amino acids.” For example, the alignment below has 37.5% sequence identity over 56 amino acids ((21 identities/56 residues)×100%), where 56 is the total length of the aligned region.

| | |
|---|---|
| RTPSDKPVAH--VANPQLQWLNRRANALLANGVE-RDNQLVV--EGLYLIYSQVLF | 56 resid. |
| \| \| \| \| \|\| \| \| \| \| \|\| \| \|\| \|\|\| \| \| \| | 21 ident. |
| RAPFKKSWAYLQVAKHKLSW-NK--DGIL-HGVRYQDGNLVIQFPGLYFIICQLQF | 56 resid. |
| (Query is SEQ ID NO:2; Sbjct is SEQ ID NO:3.) | |

Both of the sequences in the aligned region may be contained within longer, less homologous sequences. “Unrelated” or “non-homologous” sequences typically share less than 40% identity at the peptide level, preferably less than 25% identity.

Polynucleotides, DNA molecules, vectors, and such, used in the practice of the present invention may be isolated using standard cloning methods such as those described by Maniatis et al. (Molecular Cloning: A Laboratory Manual, Cold Spring Harbor, N.Y., 1982; which is incorporated herein by reference), Sambrook et al. (Molecular Cloning: A Laboratory Manual, Second Edition, Cold Spring Harbor, N.Y., 1989; which is incorporated herein by reference) or Mullis et al. (U.S. Pat. No. 4,683,195; incorporated herein by reference). Alternatively, the polynucleotides coding for polypeptides of the present invention may be synthesized using standard techniques that are well known in the art, such as by synthesis on an automated DNA synthesizer. In one embodiment of the invention, DNA sequences encoding the polypeptides of the present invention are obtained by PCR amplification using primers designed from SEQ ID NO: 1.

DNA molecules encoding the polypeptides of the present invention may be inserted into DNA constructs. As used within the context of the present invention, a DNA construct, such as an expression vector, is understood to refer to a DNA molecule, or a clone of such a molecule, either single- or double-stranded, which has been modified through human intervention to contain segments of DNA combined and juxtaposed in a manner that would not otherwise exist in nature. DNA constructs may be engineered to include a first DNA segment encoding a polypeptide of the present invention operably linked to additional DNA segments required for the expression of the first DNA segment. Within the context of the present invention, additional DNA segments will generally include promoters and transcription terminators and may further include enhancers and other elements. One or more selectable markers may also be included. DNA constructs useful for expressing cloned DNA segments in a variety of prokaryotic and eukaryotic host cells can be prepared from readily available components or purchased from commercial suppliers.

DNA constructs may also contain DNA segments necessary to direct the secretion of a polypeptide or protein of interest. Such DNA segments may include at least one secretory signal sequence. Secretory signal sequences, also called leader sequences, prepro sequences and/or pre sequences, are amino acid sequences that act to direct the secretion of mature polypeptides or proteins from a cell. Such sequences are characterized by a core of hydrophobic amino acids and are typically (but not exclusively) found at the amino termini of newly synthesized proteins. Very often the secretory peptide is cleaved from the mature protein during secretion. Such secretory peptides contain processing sites that allow cleavage of the secretory peptide from the mature protein as it passes through the secretory pathway. The choice of suitable promoters, terminators and secretory signals is well within the level of ordinary skill in the art. Expression of cloned genes in cultured mammalian cells and in E. Coli, for example, is discussed in detail in Sambrook et al. (Molecular Cloning: A Laboratory Manual, Second Edition, Cold Spring Harbor, N.Y., 1989; which is incorporated herein by reference).

Host cells containing DNA constructs are then cultured to produce the polypeptides of the present invention. The cells are cultured according to standard methods in a culture medium containing nutrients required for growth of the particular host cells. A variety of suitable media are known in the art and generally include a carbon source, a nitrogen source, essential amino acids, vitamins, minerals and growth factors. The growth medium will generally select for cells containing the DNA construct by, for example, drug selection or deficiency in an essential nutrient which is complemented by a selectable marker on the DNA construct or co-transfected with the DNA construct. Cultured mammalian cells are generally cultured in commercially available serum-containing or serum-free media. Selection of a medium appropriate for the particular host cell used is within the level of ordinary skill in the art.

Within one embodiment of the invention, the proteins of the present invention are expressed in mammalian cells. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), and DEAE-dextran mediated transfection (Ausubel et al., eds., Current Protocols in Molecular Biology, John Wiley and Sons, Inc., N.Y., 1987), which are incorporated herein by reference. Cationic lipid transfection using commercially available reagents including the Boehringer Mannheim Transfection-Reagent (N->1-(2,3-Dioleoyloxy)propyl-N,N,N-trimethyl ammoniummethylsulfate; Boehringer Mannheim, Indianapolis, Ind.) or LIPOFECTIN reagent (N->1-(2,3-dioleyloxy)propyl-N,N,N-trimethylammonium chloride and dioeleoyl phosphatidylethanolamine; GIBCO-BRL, Gaithersburg, Md.) using the manufacturer-supplied directions, may also be used. The production of recombinant proteins in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134, which are incorporated herein by reference. Preferred cultured mammalian cells include the COS-1 (ATCC No. CRL 1650), COS-7 (ATCC No. CRL 1651), BHK (ATCC No. CRL 1632), BHK 570 (ATCC No. CRL 10314) and 293 (ATCC No. CRL 1573; Graham et al., J. Gen. Virol. 36:59-72, 1977) cell lines. Additional suitable cell lines are known in the art and available from public depositories such as the American Type Culture Collection, Rockville, Md.

The recombinant polypeptides expressed using the methods described herein are isolated and purified by conventional procedures, including separating the cells from the medium by centrifugation or filtration, precipitating the proteinaceous components of the supernatant or filtrate by means of a salt, e.g. ammonium sulfate, purification by a variety of chromatographic procedures, e.g. ion exchange chromatography or affinity chromatography, or the like. Methods of protein purification are known in the art (see generally, Scopes, R., Protein Purification, Springer-Verlag, N.Y. (1982), which is incorporated herein by reference) and may be applied to the purification of the recombinant proteins of the present invention. Thus, the present invention provides the advantage that human placental bikunin or other polypeptides of the present invention may be produced having a desirable glycosylation pattern and may be readily purified using methods known in the art. Alternatively, the proteins of the present invention may be synthesized using conventional synthesis methods such as by the solid-phase synthesis such as the method of Barany and Merrifield (in The Peptides, Analysis, Synthesis, Biology Vol. 2, Gross and Meienhofer, eds, Academic Press, N.Y., pp. 1-284, 1980), by partial solid-phase techniques, by fragment condensation, or by classical solution addition.

Proteinase inhibition activity of the polypeptides of the present invention may be measured using the method essentially described by Norris et al. (Biol. Chem. Holpe-Seyler 371: 37-42, 1990). Briefly, various fixed concentrations of the polypeptide are incubated in the presence of 0.24 μg/ml of porcine trypsin (Novo Nordisk A/S, Bagsvaerd, Denmark), 12.8 CU/1 human plasmin (Kabi, Stockholm, Sweden) or 0.16 nkat/ml human plasma kallikrein (Kabi) in 100 mM NaCl, 50 mM Tris HCl, 0.01% TWEEN 80 (Polyoxyethylenesorbitan monoleate) (pH 7.4) at 25° C. After a 30 minute incubation, the residual enzymatic activity is measured by the cleavage of either of the chromogenic peptidyl nitroanilide irypsin/plasmin substrates S2251 (D-Val-Leu-Lys-Nan; Kabi) or S2302 (D-Pro-Phe-Arg-Nan; Kabi) in assay buffer. The samples are incubated for 30 minutes after which the absorbance of each sample is measured at 405 nm. An inhibition of enzyme activity is measured as a decrease in absorbance at 405 nm or fluorescence Em at 460 nm. From the results, the apparent inhibition constant K may be calculated.

In preferred embodiments, the glycosylated polypeptides (bikunins or monokunins) may be administered by any suitable route, e.g. by the oral, parenteral, bronchial, or topical routes. For such use, the compound will normally be employed in the form of a pharmaceutical composition in association with a human or veterinary pharmaceutically acceptable carrier, such as diluents, stabilizers, excipients, and or any other vehicles commonly used to formulate pharmaceutical compositions for animal or human administration. The carrier must be “acceptable” in the sense of being compatible with the other ingredients of the formulations and not deleterious to the recipient thereof. The exact form of the composition will naturally depend on the mode of administration. Actual methods for preparing parenterally administrable compositions will be known or apparent to those skilled in the art and are described in more detail in such publications as Remington's Pharmaceutical Science, 15th ed., Mack Publishing, Company, Easton, Pa. (1980).

### EXAMPLE 1

Development of Stable, High-Producing CHO Cell Lines that Express Bikunin

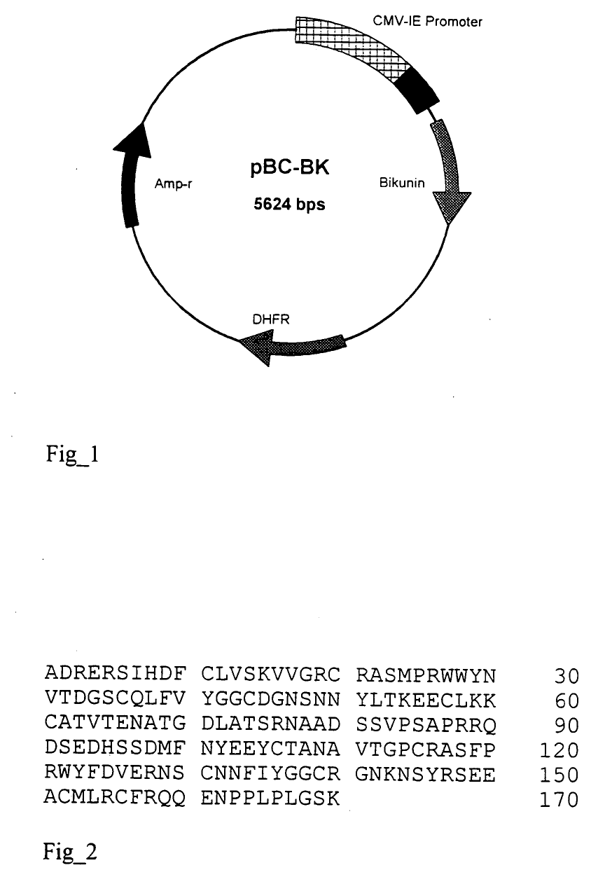

Stable production cell lines that secrete high quantities of bikunin were developed by transfecting CHO(dhfr-) cells with the expression vector shown in FIG. 1. The vector was constructed using standard recombinant DNA techniques as described in U.S. Pat. No. 5,612,213 to Chan and in Sambrook et al., 1989 (supra). The expression vector contains discrete expression cassettes for the bikunin gene (truncated bikunin—amino acid sequence given in FIG. 2) and the amplifiable and selectable gene DHFR (dihydrofolate reductase). About 1×10⁶ CHO (Chinese hamster ovary) cells were transfected with 10 μg of pBC-BK using Lipofectin reagents (Life Technology, Bethesda, Md.) according to manufacturer's instructions. The cells were then selected in the presence of 50 nM methotrexate and grown in DME/F12 media deficient in thymidine and hypoxanthine plus 5% dialyzed fetal bovine serum. Cell populations were screened for bikunin production with a chromogenic assay. Briefly, bikunin standards or culture fluid was serially diluted and incubated with an equal volume of kallikrein at 37° C. for 30 minutes after which a chromogenic substrate, N-benzoyl-Pro-Phe-Arg-pNA, was added. The reaction was incubated for 15 minutes before the addition of 50% acetic acid. The amount of p-nitroanilide released was measured at 405 nM. The high producing populations were further selected in media containing increasing concentrations of methotrexate (100 to 400 nM methotrexate) and screened for the production of bikunin. Limiting dilution cloning was then applied to derive clones with high and stable productivity. The cloning was done in the absence of methotrexate using standard tissue culture techniques by depositing 1 cell/well in 96-well plates. A clone designated FD3-1 was chosen for productivity evaluation in a bioreactor and was deposited on Nov. 12, 1999 with the American Type Culture Collection (ATCC), Rockville, Md., and was assigned accession number ______.

### EXAMPLE 2

Serum-Free Production of Bikunin In A Perfusion Bioreactor

Continuous production of bikunin was done by continuous perfusion fermentation. A 1.5-liter Wheaton fermenter was inoculated with a stable CHO cell line at 2×10⁶ cells/ml and perfused at a medium exchange rate of 0.5 liters/day. The production medium was a DME/F12-based medium supplemented with insulin (10 μg/ml) and FeSO₄.EDTA (50 μM). The cell density was maintained at 4×10⁶ cells/ml. The average daily yield of the fermenter was ˜20 mg/day. The production of bikunin was stably maintained for 21 days.

### EXAMPLE 3

Carbohydrate Analysis of Bikunin Produced From CHO Cells

Bikunin produced from CHO cells was purified using standard chromatography techniques involving ion exchange, metal chelate, and size exclusion chromatography. Briefly, clarified tissue culture fluid was diluted with water and applied to a SP-Sepharose column at pH 5.0. Eluate was concentrated by ultrafiltration and the buffer exchanged by diafiltration for anion exchange Q-Sepharose column chromatography at pH 8.2. The eluate was adjusted in NaCl and fed onto a Zn-IMAC column at pH 7.4. The Zn-IMAC flow-through was concentrated by ultrafiltration and applied to an S-200 HR column for buffer exchange and final purification.

The sialic acid content of bikunin was analyzed by incubation with sialidase in 50 mM sodium acetate buffer-, pH 5.0, for 18 hours in a capped microfuge tube. Sialic acids were separated on a Carbo Pac PA1 anion-exchange column using a buffer gradient of 20—250 mM sodium acetate in 100 mM NaOH for 50 minutes at a flow rate of 1 ml/min. Detection was done with a pulsed electrochemical detector and quantitated by comparing retention times and peak areas of samples to standard sialic acids (N-acetyneuraminic acid and N-glutaryineuraminic acid). The results were presented in Table 2.

TABLE 2
Sialic acid composition of bikunin

| Sialic acid | Contents (g/100 g of bikunin) |
|---|---|
| N-Acetylneuraminic acid | 5.4 |
| N-Glutarylneuraminic acid | 0 |

### CONCLUSION

The above examples are intended to illustrate the invention and it is thought variations will occur to those skilled in the art. Accordingly, it is intended that the scope of the invention should be limited only by the claims below.

What is claimed is:

1. A method of producing a mammalian glycosylated bikunin, the method comprising the steps of
a) obtaining mammalian cells which contain a nucleic acid coding sequence for the bikunin;
b) culturing the mammalian cells in a culture system under conditions sufficient to allow the cells to express the bikunin, said mammalian cells being capable of glycosylating the bikunin; and
c) recovering the glycosylated bikunin from the culture system.

2. An isolated mammalian glycosylated bikunin.

3. The glycosylated bikunin of claim 2, wherein the bikunin has a deduced amino acid sequence which is at least 80% identical over at least 40 residues to SEQ ID NO: 1.

4. The glycosylated bikunin of claim 2, wherein the bikunin has a deduced amino acid sequence which is at least 90% identical over at least 50 residues to SEQ ID NO: 1.

5. The glycosylated bikunin of claim 2, wherein the bikunin has a deduced amino acid sequence given by SEQ ID NO: 1.

6. The glycosylated bikunin of claim 2 wherein the glycosylated bikunin comprises at least one sialic acid residue bonded within the glycosylated bikunin via an alpha-(2, 3) linkage.

7. The glycosylated bikunin of claim 2 wherein the glycosylated bikunin comprises at least one sialic acid residue bonded within the glycosylated bikunin via an alpha-(2, 6) linkage.

8. The glycosylated bikunin of claim 2 wherein the glycosylated bikunin comprises at least one sialic acid residue bonded within the glycosylated bikunin via an alpha-(2, 3) linkage and at least one sialic acid residue bonded within the glycosylated bikunin via an alpha-(2, 6) linkage.

9. The glycosylated bikunin of claim 2 in a pharmaceutically acceptable carrier.

10. A mammalian cell line which has been engineered using recombinant DNA techniques to express a mammalian glycosylated bikunin.

11. The cell line of claim 10 wherein the cell line is a CHO cell line.

12. The cell line of claim 11 wherein the cell line is designated FD3-1 (ATCC accession number ______, deposited Nov. 12, 1999).

13. The cell line of claim 10 wherein the cell line is an HKB cell line.

14. A method of producing a mammalian glycosylated monokunin, the method comprising the steps of
d) obtaining mammalian cells which contain a nucleic acid coding sequence for the monokunin;
e) culturing the mammalian cells in a culture system under conditions sufficient to allow the cells to express the monokunin, said mammalian cells being capable of glycosylating the monokunin; and
f) recovering the glycosylated monokunin from the culture system.

15. An isolated mammalian glycosylated monokunin.

16. The glycosylated monokunin of claim 15, wherein the monokunin has a deduced amino acid sequence which is at least 80% identical over at least 40 residues to SEQ ID NO: 1.

17. The glycosylated monokunin of claim 15, wherein the monokunin has a deduced amino acid sequence which is at least 90% identical over at least 50 residues to SEQ ID NO: 1.

18. The glycosylated monokunin of claim 15 wherein the glycosylated monokunin comprises at least one sialic acid residue bonded within the glycosylated monokunin via an alpha-(2, 3) linkage.

19. The glycosylated monokunin of claim 15 wherein the glycosylated monokunin comprises at least one sialic acid residue bonded within the glycosylated monokunin via an alpha-(2, 6) linkage.

20. The glycosylated monokunin of claim 15 wherein the glycosylated monokunin comprises at least one sialic acid residue bonded within the glycosylated monokunin via an alpha-(2, 3) linkage and at least one sialic acid residue bonded within the glycosylated monokunin via an alpha-(2, 6) linkage.

21. The glycosylated monokunin of claim 15 in a pharmaceutically acceptable carrier.

22. A mammalian cell line which has been engineered using recombinant DNA techniques to express a mammalian glycosylated monokunin.

23. The cell line of claim 22 wherein the cell line is a CHO cell line.

24. The cell line of claim 22 wherein the cell line is an HKB cell line.


SEQUENCE LISTING (3 sequences)

SEQ ID NO: 1 - protein, 170 aa, human
ADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSK

SEQ ID NO: 2 - protein, 51 aa, Artificial Sequence
  Description of Artificial Sequence Random sequence
RTPSDKPVAHVANPQLQWLNRRANALLANGVERDNQLVVEGLYLIYSQVLF

SEQ ID NO: 3 - protein, 52 aa, Artificial Sequence
  Description of Artificial Sequence Random sequence
RAPFKKSWAYLQVAKHKLSWNKDGILHGVRYQDGNLVIQFPGLYFIICQLQF